(19) United States
(12) Patent Application Publication
(10) Pub. No.: US 2026/0047986 A1
(43) Pub. Date: Feb. 19, 2026

(54) SHEET-LIKE COSMETIC PRODUCT

(71) Applicant: L'OREAL, Paris (FR)

(72) Inventors: Gaurav AGARWAL, Kawasaki-shi (JP); Hiroyuki OGATA, Kawasaki-shi (JP)

(21) Appl. No.: 19/101,170

(22) PCT Filed: Jul. 7, 2023
(86) PCT No.: PCT/JP2023/025974

§ 371 Date: Feb. 4, 2025

(30) Foreign Application Priority Data
Aug. 8, 2022 (JP) 2022-126278
Oct. 10, 2022 (FR) 2210348

(51) Int. Cl. A61K 8/02; A61K 8/73; A61K 8/81; A61Q 19/00

(52) U.S. Cl. CPC A61K 8/0212; A61K 8/731; A61K 8/8129; A61Q 19/00

(57) ABSTRACT

A sheet-like cosmetic product contains (A) at least one water soluble/dispersible substrate; and (B) at least one coating layer disposed on the water soluble/dispersible substrate. The (B) at least one coating layer contains (b1) at least one clay and (b2) at least one water soluble binder. An embodiment of the product has an improved usability being easily removed from the skin after it is used.

### TECHNICAL FIELD

The present invention relates to a sheet-like cosmetic product, preferably a sheet-like cosmetic product comprising clay.

### BACKGROUND ART

Oiliness of skin is caused by excess secretion of oily/waxy matter from sebaceous glands. Sebaceous glands are found in greatest abundance on the face and scalp, though they are distributed throughout all skin sites except the palms and soles. Sebum contains triglycerides, waxes, squalene and metabolites of fat-producing cells. Although sebum keeps hair and skin supple, excess sebum can contribute to concentrating of horny cells at the opening of sebaceous follicles on the surface of the skin, this causes constriction in the follicular opening leading to a plug or comedones. This can result in blackheads, whiteheads, significant irritation, redness, tissue damage and acne.

The use of mineral clays as medicinal and cosmetic tools has been popular for a long time. Clays are used to absorb excess oil, dirt, and toxins from the skin while simultaneously exfoliating and improving skin circulation. For example: US patent 2006/0292108 describes a skin care composition composed of 10% Kaolin.

Also, clay in powder form or in ready-made paste form is sold as a clay mask or as a face pack. The term “face pack” here refers to a product in the form of a cream or paste which is applied as a mask by making a thin layer of the product on the skin for a specific period of time or until drying of the product and then is removed by rubbing or washed away using water. However, the application of such a product is a messy process and consumers need to rub the skin for removal of such a product. Upon drying of such clay products, when a consumer moves his/her facial muscles, dried mud tends to fall down from the face and soils the bathroom, clothes, etc. forcing the user to clean or to wash. In addition, in general, for post application of such clay masks, consumers also need to use a cleansing product to clean the face.

Also, WO2015/075143 reports an applicator body composition comprising at least one clay and at least one water soluble polymer, which can be provided in the form of a face mask. The product of this publication is intended to be peeled off from the skin after it is applied on the skin.

However, there is still a need to develop a clay-based sheet product which has an improved usability, in particular which can be easily removed from the skin after it is used.

### DISCLOSURE OF INVENTION

An objective of the present invention is to provide a clay-based sheet product which has an improved usability, in particular which can be easily removed from the skin after it is used. The above objective of the present invention can be achieved by a sheet-like cosmetic product, comprising
(A) at least one water soluble/dispersible substrate; and
(B) at least one coating layer disposed on the water soluble/dispersible substrate, comprising (b1) at least one clay and (b2) at least one water soluble binder.

The product may be for caring for and/or conditioning a keratinous substance, such as the skin.

The product may be a skin mask, preferably a facial mask. The (A) water soluble/dispersible substrate may comprise water soluble/dispersible polymer.

The water soluble/dispersible polymer may be a water soluble polymer selected from the group consisting of celluloses and derivatives thereof, polyvinyl alcohol, and a mixture thereof. The content of the coating layer(s) may range from 1 g/m² to 200 g/m², preferably from 5 g/m² to 100 g/m² and, more preferably from 10 g/m² to 70 g/m², and even more preferably from 15 g/m² to 50 g/m² of the product.

The coating layer(s) may comprise the (b1) clay(s) in an amount ranging from 50% to 99.5% by weight, preferably from 65% to 99% by weight, more preferably from 80% to 98% by weight, and even more preferably from 90% to 97% by weight, relative to the total weight of the coating layer(s).

The (b2) water soluble binder may be selected from the group consisting of starch, pullulan, cellulose, derivatives thereof, and a blend thereof, polyvinylalcohol, polyvinylpyrrolidone, and a mixture thereof.

The (b2) water soluble binder may be different from the water soluble/dispersible polymer included in the (A) water soluble/dispersible substrate.

The coating layer(s) may comprise the (b2) water soluble binder(s) in an amount ranging from 0.1% to 15% by weight, preferably from 0.5% to 10% by weight, more preferably from 1 to 7% by weight, and even more preferably from 2 to 5% by weight, relative to the total weight of the coating layer(s).

The coating layer may comprise at least one polyol.

The content of the polyol(s) in the coating layer(s) may range from 0.001 to 20% by weight, preferably from 0.01 to 15% by weight, more preferably from 0.1 to 10% by weight, and even more preferably from 1 to 5% by weight, relative to the total weight of the coating layer(s).

The present invention also relates to a method for manufacturing the sheet-like cosmetic product according to the present invention, comprising the steps of:
(i) preparing (A) at least one water soluble/dispersible substrate,
(ii) preparing a coating solution by mixing (b1) at least one clay and (b2) at least one water soluble binder in at least one solvent,
(iii) applying the coating solution on the (A) water soluble/dispersible substrate, and
(iv) drying the applied coating solution to form the (B) coating layer to obtain the sheet-like cosmetic product.

The solvent in step (ii) may be selected from linear or branched lower mono-alcohols, preferably linear or branched lower mono-alcohols having from 1 to 8 carbon atoms, such as ethanol, propanol, butanol, isopropanol, and isobutanol, and a combination thereof.

The present invention also relates to a non-therapeutic cosmetic process for caring for and/or conditioning a keratinous substances, such as the skin, in particular the facial skin, comprising the steps of:
(1) wetting the keratinous substance:
(2) applying the sheet-like cosmetic product according to the present invention on the wet keratinous substance; and
(3) leaving the applied product on the keratinous substance for a certain time; and
(4) rinsing off the product from the keratinous substance with water; or
(4′) peeling the product off from the keratinous substance, and
(5) rinsing off a residue of the product from the keratinous substance with water.

### BRIEF DESCRIPTION OF DRAWINGS

FIG. 1 shows one embodiment of the present invention of a facial mask.

### BEST MODE FOR CARRYING OUT THE INVENTION

After diligent research, the inventors have surprisingly discovered that a sheet-like product formed from (A) at least one water soluble/dispersible substrate; and (B) at least one coating layer disposed on the water soluble/dispersible substrate, which comprises (b1) at least one clay and (b2) at least one water soluble binder, has an improved usability being easily removed from the skin after its application, thus completing the present invention.

Thus, the product according to the present invention is a sheet-like cosmetic product, comprising:
(A) at least one water soluble/dispersible substrate; and
(B) at least one coating layer disposed on the water soluble/dispersible substrate, comprising (b1) at least one clay and (b2) at least one water soluble binder.

Hereinafter, the product, the method, the process, and the use according to the present invention will be explained in a more detailed manner.

### [Product]

The product according to the present invention is a cosmetic product, preferably a cosmetic product for a keratinous substance, and more preferably a cosmetic product for caring for and/or conditioning keratinous substances. The keratinous substance here means a material containing keratin as a main constituent element, and examples thereof include the skin, facial skin, scalp, lips, and the like. Preferably, the keratinous substance here is the skin, in particular the facial skin.

In one embodiment of the present invention, the product according to the present invention is a skin-care product, and in particular a facial skin-care product.

The product according to the present invention comprises (A) at least one water soluble/dispersible substrate and (B) at least one coating layer on the water soluble/dispersible substrate. The (B) coating layer comprises (b1) at least one clay and (b2) at least one water soluble binder. In other words, the product according to the present invention is formed from (A) at least one water soluble/dispersible substrate and (B) at least one coating layer on the water soluble/dispersible substrate, which comprises (b1) at least one clay and (b2) at least one water soluble binder. The ingredients in the product will be described in a detailed manner below.

### (A) Water Soluble/Dispersible Substrate

The product according to the present invention comprises (A) at least one water soluble/dispersible substrate as a substrate or a support. A single type of the water soluble/dispersible substrate may be used, but two or more different types of the water soluble/dispersible substrates may be used in combination.

The term “water soluble/dispersible substrate” here means a substrate in the shape of a sheet, paper, or a film and which is water soluble/dispersible. The term “water soluble/dispersible” here indicates a substance which disintegrates and/or dissolves upon immersion in water at 25° C., e.g. over period of several seconds or minutes to several days without stirring. In one embodiment, the “water soluble/dispersible” of the present invention disintegrates and/or dissolves upon immersion in water at 25° C. less than one minute.

The water-soluble/dispersible substrate may be in the form of fibers, such as woven fibers, knitted fibers, non-woven fibers and/or a polymeric mesh. The term “non-woven” here means a substrate including fibers in which the individual fibers or filaments are arranged in a disordered manner in a sheet-like structure and which are neither woven nor knit.

The water soluble/dispersible substrate is made of at least one water soluble/dispersible polymer. The water soluble/dispersible polymer may be selected from among water soluble polymers.

Preferably, the water soluble polymer may be a water-soluble film-forming polymer. Synthetic and/or natural water-soluble film-forming polymers may be used.

As the water-soluble film-forming polymer used in the present invention, mention can be made of:
polysaccharides including starches, pullulans, celluloses, derivatives thereof, and combinations thereof
proteins, such as proteins of plant origin, such as wheat or soya proteins; proteins of animal origin, such as keratins, for example keratin hydrolysates and sulphonic keratins;
acrylic polymers or copolymers, such as polyacrylates or polymethacrylates;
vinyl polymers, such as polyvinylpyrrolidones, copolymers of methyl vinyl ether and of malic anhydride, the copolymer of vinyl acetate and of crotonic acid, copolymers of vinylpyrrolidone and of vinyl acetate, copolymers of vinylpyrrolidone and of caprolactam, or polyvinyl alcohol;
anionic, cationic, amphoteric or nonionic chitin or chitosan polymers;
gums arabic, guar gum, xanthan compounds or karaya gum;
alginates and carrageenans;
glycoaminoglycans, hyaluronic acid and its compounds;
shellac resin, gum sandarac, dammars, elemis or copals;
deoxyribonucleic acid;
mucopolysaccharides, such as chondroitin sulphates;
and their mixtures.

Advantageously, the water soluble polymer used in the present invention is selected from the group consisting of celluloses and derivatives thereof, and polyvinyl alcohol, and a mixture thereof. Preferred examples of the cellulose derivatives include cellulose ethers, notably methylcellulose, hydroxyethylcellulose, hydroxypropylcellulose, hydroxypropyl methylcellulose, ethylhydroxyethylcellulose, carboxymethylcellulose and quaternized cellulose compounds.

As commercially available products of t the hydroxypropyl methylcellulose (HPMC) polymer, mention can be made of those available under the trade names METHOCEL K35, METHOCEL A15LV, METHOCEL Premium E5 LV and K35 LV from Dow chemical co.

The water soluble/dispersible substrate may additionally comprise at least one water-insoluble polymer.

The term “water insoluble” here indicates that a substance does not dissolve or readily break apart upon immersion in water.

The water-insoluble polymer may be in the form of fibers, such as woven fibers, knitted fibers, non-woven fibers or/and a polymeric mesh. Preferably, the water-insoluble polymer is in the form of non-woven fibers.

The water-insoluble polymer may be natural fibers, synthetic fibers or mixtures thereof. Non limiting examples of the synthetic fibers are polyester, polyolefin (polypropylene, polyethylene), polyamide (Nylon 6, Nylon 66), viscose, acrylic fibers, modacrylic fibers, polyvinylidene chloride and spandex. Examples of the natural fibers include cellulosic fibers (such as wood pulp, cotton, hemp, jute, and flax fibers), silk, and keratin (such as wool and camel hair fibers).

In one preferred embodiment of the present invention, the water-insoluble polymer comprises natural fibers, in particular cellulosic fibers, such as wood pulp.

As non-limiting examples of commercially available water soluble/dispersible substrates, mention can be made of water soluble paper obtainable from Nippon Paper Papilia, smartSolve, and Aquashol cooperation, and fibrous material such as nonwoven/woven materials made of PVA fiber from Kuraray etc. One example of the commercially available water soluble/dispersible substrate is water soluble paper sold under the name of 30MDP obtainable from Nippon Paper Papilia.

A thickness of the water-soluble/dispersible substrate is not particularly limited, but in general the water-soluble/dispersible substrate has a thickness ranging from 0.01 mm to 0.7 mm; more preferably from 0.02 mm to 0.5 mm, and even more preferably from 0.03 mm to 0.2 mm.

The water soluble/dispersible substrate can be made by a conventional paper making process or any kind of film casting technology.

### (B) Coating Layer

The product according to the present invention comprises (B) at least one coating layer disposed on the (A) water soluble/dispersible substrate. One coating layer may be disposed on one side of the (A) water soluble/dispersible substrate, or two coating layers may be disposed on both sides of the (A) water soluble/dispersible substrate.

Preferably, the coating layer is disposed on one side of the (A) water soluble/dispersible substrate. In this embodiment, the product of the present invention comprises one coating layer.

The content of the coating layer(s) may be 1 g/m² or more, preferably 5 g/m² or more, more preferably 10 g/m² or more, and even more preferably 15 g/m² or more, and/or may be 200 g/m² or less, preferably 100 g/m² g/m² or less, more preferably 70 g/m² or less, and even more preferably 50 g/m² g/m² or less of the sheet-like cosmetic product.

The content of the coating layer(s) may range from 1 g/m²to 200 g/m², preferably from 5 g/m² to 100 g/m², more preferably from 10 g/m² to 70 g/m², and even more preferably from 15 g/m² to 50 g/m² of the sheet-like cosmetic product.

The coating layer comprises (b1) at least one clay and (b2) at least one water soluble binder. In other words, the (b1) clay and the (b2) water soluble binder are applied, deposited or coated on the (a) water soluble/dispersible substrate. The ingredients in the coating layer will be described in a detailed manner below.

### (b1) Clay

The coating layer of the present invention comprises (b1) at least one clay. A single type of the clay may be used, but two or more different types of clay may be used in combination.

The term “clay” here refers to a naturally occurring material composed primarily of fine-grained minerals, which is generally plastic at appropriate water content and will harden when dried or fired. Although clay usually contains phyllosilicates, it may contain other materials that impart plasticity and harden when dried or fired. Associated phases in clay may include materials that do not impart plasticity and organic matter. A common definition is that in the Penguin Dictionary of Science, namely “finely divided rock materials whose component minerals are various silicates, mainly of magnesium or aluminium origin”. Clay may include Kaolinite (typically defined as [Si₄]Al₄O₁₀(OH)₈ₙH₂O (n=0 or 4)), Illite (typically defined as Mx[Si_{6.8}Al_{1.2}]Ab₃Fe_{0.025}Mg_{0.75}O₂₀(OH)₄), Vermiculite (typically defined as Mx[Si₇Al]AlFe_{0.05}Mg_{0.5}O₂(OH)₄), Smectite (typically defined as Mx[Si₈]Al_{3.2}Fe_{0.2}Mg_{0.6}O₂(OH)₄, and Chlorite (typically defined as (Al(OH)_{2.55})₄[Si_{6.8}AlO_{1.2}}A1_{3.4}Mg_{0.6})₂₀(OH)₄).

Another definition, frequently used by chemists, is “a naturally occurring sediment or sedimentary rock composed of one or more minerals and accessory compounds, the whole usually being rich in hydrated aluminum silicate, iron or magnesium, hydrated alumina, or iron oxide, predominating in particles of colloidal or near-colloidal size, and commonly developing plasticity when sufficiently pulverized and wetted” (see Kirk-Othmer, Encyclopaedia of Chemical Technology, Volume 5, page 544, 2nd edition, John Wiley and Sons, Inc., New York, New York 1964). Example of clays are given in the book “Clay mineralogy, S. Caillere, S. Henin, M. Rautureau, 2nd edition 1982, Masson”. Clays may be of natural or synthetic origin.

Hydrophilic clay includes smectites such as saponites, hectorites, montmorillonites, bentonites, beidellite. Hydrophilic clay includes synthetic hectorites (also called laponites) as the products sold by the company BYK-Chemie under the name Laporte Laponite XLG, Laponite RD, Laponite RDS (these products are sodium silicates and magnesium silicates in particular sodium, lithium and magnesium) bentonites as the product sold under the name Bentone® HC Rheox, magnesium silicates and aluminum products such as hydrated sold by Vanderbilt Company as ultra Veegum®, Veegum® HS, Veegum® DGT, or calcium silicates and particularly the synthetic form sold by the company Imerys under the name Micro-cel® C.

Fuller's earth consists chiefly of hydrated aluminum silicates that contain metal ions such as magnesium, sodium, and calcium within their structure. Montmorillonite is the principal clay mineral in fuller's earth, but other minerals such as kaolinite, attapulgite, and palygorskite among other components can also be included.

Lipophilic clay means clay swellable in a lipophilic medium, the clay swells and forms a colloidal dispersion. Lipophilic clays include modified clays such as the modified magnesium silicate (Bentone gel VS38 from Rheox) hectorites modified with an ammonium chloride fatty acid Cia to C22, as hectorite modified with ammonium chloride distearyldimethylammonium (CTFA name: Disteardimonium hectorite) sold under the name “Bentone 38 CE” by Rheox or Bentone®38V by ELEMENTIS.

The origin of such clay can be natural or synthetic mineral clay such as hectorite, bentonite, and quaternized derivatives thereof, for example which are obtained by reacting the minerals with a quaternary ammonium compound, such as stearalkonium bentonite, hectorites, quaternized hectorites such as Quatemium-18 hectorite, carbonates such as propylene carbonate, bentones, and the like.

Non-limiting examples of clay which can be used in the present invention are Fuller's earth, Pinatubo volacanic ash mud in the Philippines, Aleppo clay from Syria, Pulau tiga volcano mud from Malaysia, Nha Trang mud from Vietnam, White Kaolinite from Korea, Yellow Loess from Korea, Jeju volcanic clay from Korea, Guanziling mud from Taiwan, Wudalianchi volcanic mud from China, Black mud of Yuncheng salt lake from China, mineral mud from Tantou village in China, China clay (Kaolin), Maifan stone from China, Beppu onsen Fango from Japan, Kucha from Japan, Tanakura clay from Japan, Cambrian blue clay from Russia, Blue Lagoon mud from Iceland, Saki lake mud from Ukraine, Karlovy vary moor mud from Czech Republic, Heviz Georgikon moor mud from Hungary, Alpine moor mud from Austria, Bad wilsnack mud from Germany, Bavarian mineral slat mountain mud from Germany, Freiburg volcanic ash from Germany, Santorini mud from Greece, Mar menor mud from Spian, Ischian volcanic mud from Italy, Euganean thermal mud from Italy, Yellow clay-lllite from France, French Green Clay—Montmorrillonite, Calistoga mud from the U.S.A., Sacred clay and ormalite from the U.S.A., Redmond clay from the U.S.A., Arctic mineral mud from Canada, Tulum Mayan clay from Mexico, Glacial clay from Canada, Amazonian white clay from Brazil, El Chillante volcanic thermal mud from Argentina, African healing clay, and Australian olive green clay.

The content of the clay(s) in the coating layer(s) of the present invention may be 50% by weight or more, preferably 65% by weight or more, more preferably 80% by weight or more, and even more preferably 90% by weight or more, and/or may be in general 99.5% by weight or less, preferably 99% by weight or less, more preferably 98% by weight or less, and even more preferably 97% by weight or less, relative to the total weight of the coating layer(s). Any combinations of the values of the upper and lower limit are available.

The coating layer(s) of the present invention may comprise the clay(s) in an amount ranging from 50% to 99.5% by weight, preferably from 65% to 99% by weight, more preferably from 80% to 98% by weight, and even more preferably from 90% to 97% by weight, relative to the total weight of the coating layer(s).

### (b2) Water Soluble Binder

50 The coating layer of the present invention comprises (b2) at least one water soluble binder. A single type of the water soluble binder may be used, but two or more different types of the water soluble binders may be used in combination.

The water soluble binder can function to adhere the (b1) clay to the coating layer, in particular to adhere the (b1) clay to the (A) water soluble/dispersible substrate.

In one preferred embodiment, the (b2) water soluble binder comprises at least one polar polymer. In another preferred embodiment, the (b2) water soluble binder is selected from water-soluble film-forming polymers. As the water-soluble film-forming polymer used in the present invention, mention can be made of:
polysaccharides including starches, pullulans, celluloses, derivatives thereof, and combinations thereof
proteins, such as proteins of plant origin, such as wheat or soya proteins; proteins of animal origin, such as keratins, for example keratin hydrolysates and sulphonic keratins;
acrylic polymers or copolymers, such as polyacrylates or polymethacrylates;
vinyl polymers, such as polyvinylpyrrolidones, copolymers of methyl vinyl ether and of malic anhydride, the copolymer of vinyl acetate and of crotonic acid, copolymers of vinylpyrrolidone and of vinyl acetate, copolymers of vinylpyrrolidone and of caprolactam, or polyvinyl alcohol;
anionic, cationic, amphoteric or nonionic chitin or chitosan polymers;
gums arabic, guar gum, xanthan compounds or karaya gum;
alginates and carrageenans;
glycoaminoglycans, hyaluronic acid and its compounds;
shellac resin, gum sandarac, dammars, elemis or copals;
deoxyribonucleic acid;
mucopolysaccharides, such as chondroitin sulphates;
and their mixtures.

Advantageously, the water-soluble film-forming polymer is selected from the group consisting of starch, pullulan, starch, cellulose, derivatives thereof, and blend thereof, polyvinylalcohol, polyvinylpyrrolidone, and a mixture thereof.

The (b2) water soluble binder may be the same as or different from the water soluble/dispersible polymer forming the (a) water soluble/dispersible substrate. These materials can be distinguished each other in consideration of their functions and/or states. Specifically, the water soluble/dispersible polymer in the (a) water soluble/dispersible substrate forms the substrate, i.e., this polymer is a part of the substrate, while the (b2) water soluble binder acts to adhere the (b1) clay to the substrate. A skilled person can easily distinguish them by analyzing the product of the present invention. For example, the (b2) water soluble binder is directly contact with the (b1) clay and can be present at the surface of the (b1) clay. On the other hand, the water soluble/dispersible polymer in the (a) water soluble/dispersible substrate is positioned within the substrate.

In one embodiment of the present invention, the (b2) water soluble binder is different from the water soluble/dispersible polymer forming the (a) water soluble/dispersible substrate.

The content of the water soluble binder(s) in the coating layer(s) of the present invention may be 0.1% by weight or more, preferably 0.5% by weight or more, more preferably 1% by weight or more, and even more preferably 2% by weight or more, and/or may be in general 15% by weight or less, preferably 10% by weight or less, more preferably 7% by weight or less, and even more 50 preferably 5% by weight or less, relative to the total weight of the coating layer(s). Any combinations of the values of the upper and lower limit are available.

The coating layer(s) of the present invention may comprise the water soluble binder(s) in an amount ranging from 0.1% to 15% by weight, preferably from 0.5% to 10% by weight, more preferably from 1 to 7% by weight, and even more preferably from 2 to 5% by weight, relative to the total weight of the coating layer(s).

### (Other Ingredients)

The coating layer(s) of the present invention may comprise optional ingredients other than the (b1) clay and the (b2) water soluble binder.

### Cosmetically Active Ingredient

The coating layer of the present invention may comprise at least one cosmetically active ingredient. A single type of the cosmetically active ingredient may be used, but two or more different types of the cosmetically active ingredients may be used in combination.

The cosmetically active ingredient may be a skin care agent. As the skin-care agents, mention can be made of whitening agents, brightening agents, anti-oxidants, cleansing agents, free radical scavengers, moisturizers, skin tone altering agents, anti-acne agents, anti-aging agents, anti-wrinkle agents, anti-inflammatory agents, skin texture treatment agents, anti-perspirant agents, aesthetics, anti-bacterial agents, cooling actives, sweat absorption actives, nourishing agents, sebum, an agent for oil absorption, and moisture absorbers and any combination thereof.

Non-limiting examples of skin whitening or brightening agents include, but are not limited to, skin tone changing pigments, reflective particulate materials, soft focus agents and any mixture thereof.

The skin tone altering agents may be selected from skin tone changing pigments including, but not limited to, talc, mica, silica, titanium oxide, zinc oxide and or titanium oxide coated mica, borosilicates; hydrophobically modified zinc oxides; hydrophobically modified titanium di-oxides, and composites of titanium-zinc oxides.

Examples of the anti-oxidants, the anti-inflammatory and/or cooling actives and/or sweat absorption actives to be used in the invention include, but are not limited to, Vitamin C, Vitamin E, Derivatives of Vitamin C and Vitamin D, Lycopene, Carrotene, Lecithin, Rice bran lipids, Rice bran oils, menthol; yoghurt, silicated menthols, menthyl lactates; almond oil; peanut oil; sunflower oil; horse gram powder; olive oil; fumated or non-fumated silica, hydrophobically modified silica, hydrophylically modified silica and any mixture/composite thereof.

The amount of the cosmetically active ingredient(s) in the coating layer(s) of the present invention is not particularly limited, but in general may range from 0.01% to 10% by weight, preferably from 0.05% to 5% by weight, and more preferably from 0.1 to 3% by weight, relative to the total weight of the coating layer(s). In one embodiment of the present invention, the coating layer is free of cosmetically active ingredients.

### Polyol

The coating layer of the present invention may comprise at least one polyol. A single type of the polyol may be used, but two or more different types of the polyols may be used in combination.

The term “polyol” here means an alcohol having two or more hydroxy groups, and does not encompass a saccharide or a derivative thereof. The derivative of a saccharide includes a sugar alcohol which is obtained by reducing one or more carbonyl groups of a saccharide, as well as a saccharide or a sugar alcohol in which the hydrogen atom or atoms in one or more hydroxy groups thereof has or have been replaced with at least one substituent such as an alkyl group, a hydroxyalkyl group, an alkoxy group, an acyl group or a carbonyl group.

The polyol in the present invention may function as a plasticizer which increases the flexibility and strength of the product and in some cases the polyols can provide skin conditioning benefits.

The polyol may be a C2-C24 polyol, preferably a C2-C9 polyol, comprising at least 2 hydroxy groups, and preferably 2 to 5 hydroxy groups. The polyol may be selected from glycerins and derivatives thereof, and glycols and derivatives thereof. The polyol may be selected from the group consisting of glycerin, diglycerin, polyglycerin, ethylene glycol, diethylene glycol, propylene glycol, dipropylene glycol, butylene glycol, pentylene glycol, hexylene glycol, C6-C24 polyethyleneglycol, 1,3-propanediol, 1,4-butanediol, and 1,5-pentanediol, dextrins, and sorbitols.

In one preferred embodiment, the polyols are selected from glycerin, glycols such as ethylene glycol, propylene glycol, butylene glycols and diethylene glycols, dextrins, and sorbitols.

The content of the polyol(s) in the coating layer(s) of the present invention is not particularly limited, but in general may range from 0.001 to 20% by weight, preferably from 0.01 to 15% by weight, more preferably from 0.1 to 10% by weight, and even more preferably from 1 to 5% by weight, relative to the total weight of the coating layer(s). In one embodiment of the present invention, the coating layer is free of polyols.

### Dyes

The coating layer of the present invention may comprise at least one dye. A single type of the dye may be used, but two or more different types of the dyes may be used in combination.

The dyes which can be used in the present invention may be inorganic or organic dyes. Preferably, the dye comprises at least one inorganic dye, such as pigments and lakes. As the dye, mention can be made of titanium dioxide, yellow iron oxide, red iron oxide, black iron oxide, and caramel, carmine, carbon black, D & C Red 7 Lake, FD & C Yellow 5 Lake, FD &C Yellow dye, FD & C Blue 1 dye.

The amount of the dye(s) in the coating layer(s) of the present invention is not particularly limited, but in general may range from 0.001% to 10% by weight, preferably from 0.005% to 5% by weight, and more preferably from 0.01 to 1% by weight, relative to the total weight of the coating layer(s). In one embodiment of the present invention, the coating layer is free of the dyes.

### Optional Additives

The coating layer of the present invention may also comprise any optional additive(s) usually used in the field of cosmetics, chosen, for example, from anionic, cationic, nonionic or amphoteric surfactants, dispersants, fragrances, neutralizers, antiseptics, chelating agents, and mixtures thereof.

In one embodiment of the present invention, the product according to the present invention is a skin-care product, and in particular a facial skin-care product. In one embodiment, the sheet-like cosmetic product is for treating or caring for oily skin, for example, treating or caring for oily facial skin.

In particular, the product according to the present invention may be applied on wet surface of the skin and absorbs impurities, sebum and excess oil from the skin.

In one embodiment, the product is for mattifying the skin.

In one embodiment, the product is for relaxing cosmetic care.

In one embodiment, the product is for refreshing skin care. In addition, the present invention can also provide effects of oil control of keratinous substances, black head removal and/or pore reduction of the keratinous substances, and imparting brightness of the keratinous substances.

The functionality of the product of the present invention mainly depends on the activities of the active ingredients. Moisturization, whitening and anti-aging are the most common functionalities for such a product, especially a facial mask or a facial pad.

Furthermore, other skincare active ingredients for cosmetic benefits such as for example whitening, anti-wrinkles etc. can be present or added to the coating layer(s), which may be released to the skin during the application time.

The sheet-like cosmetic product of the present invention generally has a thickness ranging from 0.01 mm to 1 mm; more preferably from 0.05 mm to 0.5 mm, and even more preferably from 0.07 mm to 0.12 mm.

The product can have a desired shape depending the purpose of the use. The most common format of the product distribution is folded, packed in a single use sachet. In some cases, the products are distributed in the format of a roll pack. Such a roll pack comprises a substrate roll with a plurality of products of a predetermined shape, for example a plurality of facial masks. The product can be applied on such an applicator body after its detaching from the roll pack or any other packaging.

The shape of the product depends on its targeted functionality and targeted zones. One example of use of the product can be in the form of a facial mask or a facial pad, which can be cut in the shape of face with slits for eyes, mouth and nose. The products are for example selected among face masks, wipes and pads.

In one embodiment, the product is a skin mask.

In one embodiment, the product is a facial mask.

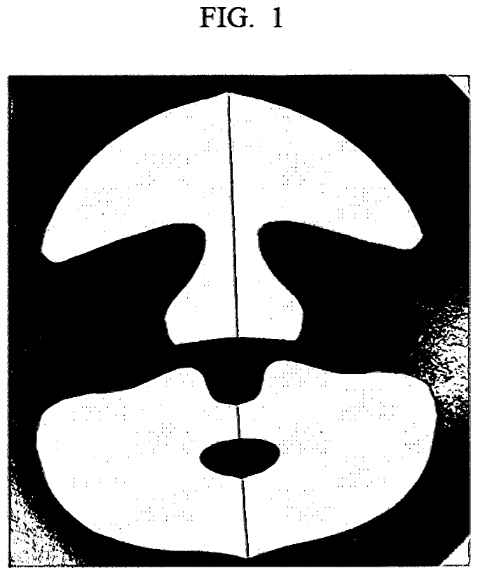

FIG. 1 represents one embodiment of the present invention, in which a facial mask is made of two pieces, prior to use. In this embodiment, the facial mask can be cut in two pieces: upper piece and lower piece in order to facilitate the easier application onto the upper face and the lower face, respectively. Such two pieces face mask presents an upper piece intended to be placed on the 50 upper part of the face and a lower piece intended to be placed on the lower part of the face.

### [Manufacturing Method]

The present invention also relates to a method for preparing the sheet-like cosmetic product according to the present invention.

Preferably, the method for preparing the sheet-like cosmetic product according to the present invention comprises the steps of:
(i) preparing (A) at least one water soluble/dispersible substrate,
(ii) preparing a coating solution by mixing (b1) at least one clay and (b2) at least one water soluble binder in at least one solvent,
(iii) applying the coating solution on the (A) water soluble/dispersible substrate, and
(iv) drying the applied coating solution to form the (B) coating layer to obtain the sheet-like cosmetic product.
Step (i) is a step of preparing (a) at least one water soluble/dispersible substrate. The (a) water soluble/dispersible substrate is the same as that explained in the “Product” part above, and thus the same explanations can be applied to the (a) water soluble/dispersible substrate here.
Step (ii) is a step of preparing a coating solution by mixing (b1) at least one clay and (b2) at least one water soluble binder in at least one solvent. The (b1) at least one clay and the (b2) at least one water soluble binder are the same as those explained in the “Product” part above, and thus the same explanations can be applied to the (b1) at least one clay and the (b2) at least one water soluble binder here.

The solvent used in the step (ii) is not particularly limited, but preferably is selected from solvents other than water. Non-limiting examples of the solvent includes, for example, linear or branched lower mono-alcohols, preferably linear or branched lower mono-alcohols having from 1 to 8 carbon atoms, such as ethanol, propanol, butanol, isopropanol, and isobutanol, and a combination thereof.

The amount of the clay(s) in the coating solution may be 1% by weight or more, preferably 5% by weight or more, more preferably 10% by weight or more, and even more preferably 20% by weight or more, relative to the total weight of the solution. The amount of the clay(s) in the coating solution may be 60% by weight or less, preferably 55% by weight or less, more preferably 50% by weight or less, and even more preferably 45% by weight or less, relative to the total weight of the solution. Any combinations of the values of the upper and lower limit are available.

The coating solution may comprise the clay(s) in an amount ranging from 1% to 60% by weight, preferably from 5% to 55% by weight, more preferably from 10% to 50% by weight, and even more preferably from 20% to 45% by weight, relative to the total weight of the solution.

The amount of the water soluble binder(s) in the coating solution may be 0.1% by weight or more, preferably 0.3% by weight or more, more preferably 0.5% by weight or more, and even more preferably 0.7% by weight or more, relative to the total weight of the solution. The amount of the water soluble binder(s) in the coating solution may be 20% by weight or less, preferably 15% by weight or less, more preferably 10% by weight or less, and even more preferably 5% by weight or less, relative to the total weight of the solution. Any combinations of the values of the upper and lower limit are available.

The coating solution may comprise the water soluble binder(s) in an amount ranging from 0.1% to 20% by weight, preferably from 0.3% to 15% by weight, more preferably from 0.5% to 10% by weight, and even more preferably from 0.7% to 5% by weight, relative to the total weight of the solution.

In one embodiment of the present invention, the amount of the polyol(s) in the coating solution may be 0.1% by weight or more, preferably 0.5% by weight or more, more preferably 0.5% by weight or more, and even more preferably 1.5% by weight or more, relative to the total weight of the solution. The amount of the polyol(s) in the coating solution may be 20% by weight or less, preferably 15% by weight or less, more preferably 10% by weight or less, and even more preferably 5% by weight or less, relative to the total weight of the solution. Any combinations of the values of the upper and lower limit are available.

In one embodiment of the present invention, the coating solution may comprise the polyol(s) in an amount ranging from 0.1% to 20% by weight, preferably from 0.5% to 15% by weight, more preferably from 1% to 10% by weight, and even more preferably from 1.5% to 5% by weight, relative to the total weight of the solution.

In one embodiment of the present invention, the coating solution includes water of less than 5% by weight, preferably less than 3% by weight, more preferably less than 1% by weight, and even more preferably less than 0.5% by weight, and in particular less than 1% by weight, relative to the total weight of the coating solution. In another embodiment of the present invention, the coating solution is anhydrous.

Step (ii) may comprise adding one or more optional ingredients, such as the cosmetically active ingredients, the polyols, the dyes, and/or optional additives as explained above, to the coating solution. Therefore, the coating solution may comprise one or more optional ingredients selected from the cosmetically active ingredients, the polyols, the dyes, and optional additives.

The method for mixing the ingredients is not particularly limited, and a conventional mixing means can be employed, such as mixing with a stirrer and/or a homogenizer.

Step (iii) is a step of applying the coating solution on the (A) water soluble/dispersible substrate. The method for applying the coating solution on the (A) water soluble/dispersible substrate is not particularly limited, and any conventional method can be employed. For example, the coating solution can be applied on the substrate using any wet coating process, such as dip coating, knife coating, bar coating, curtain coating, and screen printing.

Preferably, the coating solution is applied to both sides of the (A) water soluble/dispersible substrate, so that the product of the present invention comprises two coating layers.

Step (iv) is a step of drying the applied coating solution to form the (B) coating layer and thus obtaining the sheet-like cosmetic product. The drying temperature of the coating layer may in general range between 40° C. to 200° C., preferably between 40° C. to 100° C., and more preferably between 40° C. to 70° C. The drying time of the coating layer may in general range between a few seconds, for example one second, two seconds, three seconds, four seconds, or five seconds and 30 minutes, preferably few seconds and 15 minutes, and more preferably between a few seconds and 5 minutes.

The dried product of the present invention may be shaped according to the appropriate desired shape.

Shaping may be done by cutting the sheet-like product in the desired shape.

### [Cosmetic Process and Use]

The present invention also relates to a non-therapeutic process or use, preferably a non-therapeutic cosmetic process or use, and more preferably a cosmetic process or use for caring for and/or conditioning keratinous substances, such as the skin, in particular facial skin.

In one embodiment of the present invention, the present invention relates to a cosmetic process for caring for and/or conditioning keratinous substances, such as the skin, in particular the facial skin, comprising the steps of:
(1) wetting the keratinous substance:
(2) applying the sheet-like cosmetic product of the present invention on the wet keratinous substance; and
(3) leaving the applied product on the keratinous substance for a certain time; and
(4) rinsing off the product from the keratinous substance with water; or
(4′) peeling the product off from the keratinous substance, and
(5) rinsing off a residue of the product from the keratinous substance with water.

In another embodiment of the present invention, the present invention relates to a use of the sheet-like cosmetic product for caring for and/or conditioning keratinous substances, such as the skin, in particular the facial skin, comprising the steps of:
(1) wetting the keratinous substance:
(2) applying the sheet-like cosmetic product of the present invention on the wet keratinous substance; and
(3) leaving the applied product on the keratinous substance for a certain time; and
(4) rinsing off the product from the keratinous substance with water; or.
(4′) peeling the product off from the keratinous substance, and
(5) rinsing off a residue of the product from the keratinous substance with water.

Step (1) is a step of wetting or moistening the keratinous substance to be cared for or conditioned to make the keratinous substance wet. For this purpose, any format of a composition comprising water can be applied to the keratinous substance, for example, an aqueous solution, a toner, a micellar water, a lotion, a milky lotion, a paste, a serum, a suspension, a dispersion, an emulsion, and the like. The composition may include cosmetically acceptable ingredients or cosmetically active ingredients as mentioned above.

In one embodiment of the present invention, the composition to be applied on the keratinous substance in step (1) is water. In this embodiment, step (1) is a step of applying water on the keratinous substance to be cared for or conditioned.

Step (2) is a step of applying the sheet-like cosmetic product of the present invention on the wet keratinous substance which was treated in step (1). This step can be conducted by, for example, placing the sheet-like cosmetic product on the keratinous substance which was wetted in step (1).

Step (3) is a step of leaving the applied product on the keratinous substance for a certain time in order to make the product dry. This step can be a step for waiting a sufficient period of time, called “application time”, after placing said product according to the present invention in contact with the keratinous substance, until said product has naturally dried.

The leaving time in step (3) may range from 1 minute to 30 minutes, preferably from 3 minutes to minutes, more preferably from 5 minutes to 20 minutes, and even more preferably from 10 minutes to 20 minutes. Step (3) can be carried out at room temperature (25° C.) and atmospheric pressure (105 Pa).

In step (3), the product absorbs impurities, sebum and excess oil from the keratinous substance.

In one embodiment of the invention, the product applied on the keratinous substance is removed by rinsing off with water from the keratinous substance in Step (4) after Step (3). Because the sheet-like cosmetic product of the present invention is made of (A) at least one water soluble/dispersible substrate; and (B) at least one coating layer disposed on the water soluble/dispersible substrate, comprising (b1) at least one clay and (b2) at least one water soluble binder. For example, the product can be rinsed off by rubbing it or washing it off with the hands using water.

In another embodiment of the present invention, the product applied on the keratinous substance is peeled off from the keratinous substance in Step (4′) after Step (3). Then a residue of the product remained on the keratinous substance is rinsed off with water in Step (5). The residue of the product can be rinsed off by rubbing it or washing it off with the hands using water.

### EXAMPLES

The present invention will be described in more detail by way of examples which however should not be construed as limiting the scope of the present invention. The examples were performed at room temperature (25° C.) and atmospheric pressure (105 Pa), unless otherwise mentioned.

### Examples 1 and 2

The sheet-like cosmetic products according to the present invention were prepared in accordance with the following protocol. First, a water soluble substance in the form of paper 30MDP (thickness: 0.065 mm) obtained from Nippon Paper Papilia was prepared. Coating solutions were then prepared by mixing Kaolin, polyvinylpyrrolidone, isopropyl alcohol, and glycerin, if present, in the formulation ratio shown in Table 1 with a homogenizer. The numerical values shown in Table 1 are all based on “% by weight” as active raw materials. Each of the obtained coating solutions was applied on the water soluble substance by a bar coating process on one side of the substrate. The substrate was then dried at 70° C. for several seconds to obtain the sheet-like cosmetic products of the present invention. The amount of the coating layer was 30 g/m² of the sheet-like product. The thickness of the sheet-like product was about 0.1 mm.

TABLE 1

Formulation of Coating Solution

| Ingredients | Example 1 | Example 2 |
|---|---|---|
| Kaolin | 40 | 38 |
| Polyvinylpyrrolidone | 1.5 | 1 |
| Isopropyl Alcohol | 58.5 | 59 |
| Glycerin | — | 2 |

The obtained sheet-like product was cut into the shape of a facial mask as shown in FIG. 1. Three experts used the product in accordance with the following process:
(1) wetting their facial skin with water;
(2) applying the sheet-like cosmetic product in the form of the facial mask on the wet face;
(3) leaving the applied product on the face for 15 minutes; and
(4) rinsing off the product from the face by rubbing it with the hands using water.

The products could be easily removed from their faces by only rubbing them with the hands using water. Excess sebum and oil could be successfully removed from their faces. Also, at least the mattifying effect could be confirmed.

1. A sheet-like cosmetic product, comprising:
(A) at least one water soluble/dispersible substrate; and
(B) at least one coating layer disposed on the water soluble/dispersible substrate, comprising (b1) at least one clay and (b2) at least one water soluble binder.

2. The product according to claim 1, wherein the product is for caring for and/or conditioning a keratinous substance.

3. The product according to claim 1, wherein the product is a skin mask.

4. The product according to claim 1, wherein the (A) water soluble/dispersible substrate comprises a water soluble/dispersible polymer.

5. The product according to claim 4, wherein the water soluble/dispersible polymer is a water soluble polymer selected from the group consisting of celluloses and derivatives thereof, polyvinyl alcohol, and a mixture thereof.

6. The product according to claim 1, wherein the content of the coating layer(s) ranges from 1 g/m² to 200 g/m²of the product.

7. The product according to claim 1, wherein the coating layer(s) comprises the (b1) clay(s) in an amount ranging from 50% to 99.5% by weight, relative to the total weight of the coating layer(s).

8. The product according to claim 1, wherein the (b2) water soluble binder is selected from the group consisting of starch, pullulan, cellulose, derivatives thereof, and blend thereof, polyvinylalcohol, polyvinylpyrrolidone, and a mixture thereof.

9. The product according to claim 4, wherein the (b2) water soluble binder is different from the water soluble/dispersible polymer included in the (A) water soluble/dispersible substrate.

10. The product according to claim 1, wherein the coating layer(s) comprises the (b2) water soluble binder(s) in an amount ranging from 0.1% to 15% by weight relative to the total weight of the coating layer(s).

11. The product according to claim 1, wherein the coating layer comprises at least one polyol.

12. The product according to claim 11, wherein the content of the polyol(s) in the coating layer(s) ranges from 0.001 to 20% by weight relative to the total weight of the coating layer(s).

13. A method for manufacturing the sheet-like cosmetic product according to claim 1, comprising the steps of:
(i) preparing (A) at least one water soluble/dispersible substrate,
(ii) preparing a coating solution by mixing (b1) at least one clay and (b2) at least one water soluble binder in at least one solvent,
(iii) applying the coating solution on the (A) water soluble/dispersible substrate, and
(iv) drying the applied coating solution to form the (B) coating layer to obtain the sheet-like cosmetic product.

14. The method according to claim 13, wherein the solvent in the step (ii) is selected from linear or branched lower mono-alcohols.

15. A non-therapeutic cosmetic process for caring for and/or conditioning keratinous substances, comprising the steps of:
(1) wetting the keratinous substance:
(2) applying the sheet-like cosmetic product according to claim 1 on the wet keratinous substance; and
(3) leaving the applied product on the keratinous substance for a certain time; and
(4) rinsing off the product from the keratinous substance with water; or
(4′) peeling the product off from the keratinous substance, and
(5) rinsing off a residue of the product from the keratinous substance with water.
